(19) United States
(12) Patent Application Publication
(10) Pub. No.: US 2017/0112522 A1
(43) Pub. Date: Apr. 27, 2017

(54) DISPOSABLE SWING SCAPEL WITH REUSABLE HANDLE AND BLADE CONFIGURED FOR SCALPEL

(71) Applicants: Joseph HUTCHISON, Lynchburg, VA (US); Calvin DUDLEY, Lynchburg, VA (US)

(72) Inventors: Joseph HUTCHISON, Lynchburg, VA (US); Calvin DUDLEY, Lynchburg, VA (US)

(21) Appl. No.: 15/315,465

(22) PCT Filed: Jun. 2, 2015
(86) PCT No.: PCT/US15/33750

§ 371 Date: Dec. 1, 2016

Related U.S. Application Data
(60) Provisional application No. 62/006,499, filed on Jun. 2, 2014.

(51) Int. Cl. A61B 17/3213; A61B 17/3215

(52) U.S. Cl. CPC A61B 17/3213; A61B 17/3215

(57) ABSTRACT

A cutting device including a blade with a first end with a cutting surface and a second end configured to be attached to a handle, wherein the blade can be attached to the handle without a user contacting the blade; and wherein the blade is configured to be locked in place in they handle by rotating said handle.

### CROSS-REFERENCE TO RELATED APPLICATION

This application claims priority from U.S. Provisional Application No. 62/006,499 filed Jun. 2, 2014, the disclosure of which is incorporated herein by reference.

### BACKGROUND

1. Field

Exemplary embodiments relate to a cutting device and, in particular, to a surgical scalpel having a safer and newly designed detachable blade.

2. Description of the Related Art and Background

Scalpels are regularly used by surgeons and other healthcare professionals for making incisions in a patient during an operative procedure. Typically, a certified surgical scrub technician affixes the blade to a reusable handle at the beginning of an operation. At the end of the operation, typically the blade tang has to be grasped by the user's hand or by a clamp and pulled from the handle. This procedure is not desirable because it exposes the person attempting to remove the blade to the risk of being cut if the user's hand is used to grasp the blade tang. Even if a clamp is used, this procedure is cumbersome at best.

This awkward hand motion can lead to potential nicks or cuts that are uncomfortable and distracting at best. In addition, they may result in blood or body fluid exposure between the patient and the surgeon or other healthcare professionals in the operating room or elsewhere in the hospital. This may lead to the spread of infectious diseases between the patient and the healthcare professional. Concern over this situation has become especially acute because of such diseases as acquired immune deficiency syndrome, i.e. AIDS, and hepatitis.

While surgical gloves aid in reducing the chances of being cut during a surgical procedure, these gloves are not foolproof. And even when two sets of gloves are utilized, full protection is not afforded to the healthcare provider because the scalpel blade can still cut through both sets of gloves. Also, utilizing two sets of gloves reduces finger dexterity by the surgeon and thus is distracting to the surgeon and can interfere with the intended surgical procedure.

In view of the need for a surgical scalpel that can prevent or at least minimize the chances of accidental nicks or cuts during assembly/disassembly, numerous surgical scalpels have been designed. See for example, See also U.S. Pat. No. 5,683,407. Unfortunately, these designs are deficient because they are cumbersome, difficult to use, and may cause unwanted shielding or exposure of the scalpel blade prior to the need for it. In addition, these prior designs are deficient because they do not provide a safe and effective way for the blade to be attached and removed from the handle before or after the surgical procedure.

### SUMMARY OF THE INVENTION

Exemplary embodiments of the invention relate to a cutting device and, in particular, to a surgical scalpel having a newly designed safer detachable blade that will protect healthcare professionals from being inadvertently nicked or cut when the surgical scalpel is handled. In addition, this invention relates to a unique locking mechanism for attaching and removing the blade to and from the handle out of and into the unique blade holder pack for safer disposal either during or at the end of the surgical procedure. Moreover, this invention combines all of the above features in a surgical scalpel and handle that may be disassembled to facilitate cleaning and sterilization of the surgical scalpel handle for reuse. Finally, this invention includes a locking mechanism that prevents inadvertent partial disassembly of the surgical scalpel prior to the need to clean and sterilize the device.

One embodiment of the invention includes cutting device, with a blade with a first end with a cutting surface and a second end configured to be attached to a handle, wherein the blade can be attached to the handle without a user contacting the blade; and wherein the blade is configured to be locked in place in the handle by rotating the handle.

In other embodiments of the invention, the second end of the blade includes an opening in which the handle can be inserted.

In other embodiments of the invention, the second end of the blade includes a notch which can be used to lock the blade in said handle.

In other embodiments of the invention, the cutting device further includes a handle.

In other embodiments of the invention, the handle includes a lever configured to lock in place the blade in the handle.

In other embodiments of the invention, the lever includes an elastic member.

In other embodiments of the invention, lever includes a locking mechanism configured to lock in place the blade in the handle.

### BRIEF DESCRIPTION OF THE DRAWINGS

These and/or other aspects will become apparent and more readily appreciated from the following description of exemplary embodiments, taken in conjunction with the accompanying drawings of which:

FIG. 1 is a side view of an exemplary embodiment of the scalpel e present invention with a blade affixed.

FIG. 2 is a side view of an exemplary embodiment of the scalpel of the present invention with the handle darkened to show its relative position to the other working parts.

FIG. 3 is a side view of an exemplary embodiment of the scalpel of the present invention with the lever arm darkened to show its relative position to the other working parts.

FIG. 4 is a side view of an exemplary embodiment of the scalpel of the present invention with the spring arm darkened to show its relative position to the other working parts.

FIG. 5 is a side view of an exemplary embodiment of the scalpel of the present invention with the butt plate darkened to show its relative position to the other working parts.

FIG. 6 is a side view of an exemplary embodiment of the scalpel of the present invention with the cam-pin, retaining pin and butt plate fixation screws darkened to show relative position to the other working parts.

FIG. 7 is a side view of an exemplary embodiment of the scalpel of the present invention with the uniquely designed blade darkened to show its relative position to the other working parts.

FIG. 8 is a top view of an exemplary embodiment of the scalpel of the present invention with different materials for handles demonstrating changes in the width of the scalpel handle dependent upon manufacturing requirements.

FIG. 9 is a view of an exemplary embodiment of the packaging for the scalpel blade of the present invention.

FIGS. 10A, 10B and 10C are side views of the exemplary packaging.

FIG. 11 shows an example of how the blade is attached to the handle.

FIG. 12 shows side views of the closed packaging and views of the open packaging.

FIG. 13 shows perspective views of closed and open packaging with the packaging mounted in a block.

### DETAILED DESCRIPTION

Reference will now be made in detail to exemplary embodiments, examples of which are illustrated in the accompanying drawings, wherein like reference numerals refer to like elements throughout. However, known functions associated with the exemplary embodiments or detailed descriptions on the configuration and other matters which would unnecessarily obscure the present disclosure will be omitted.

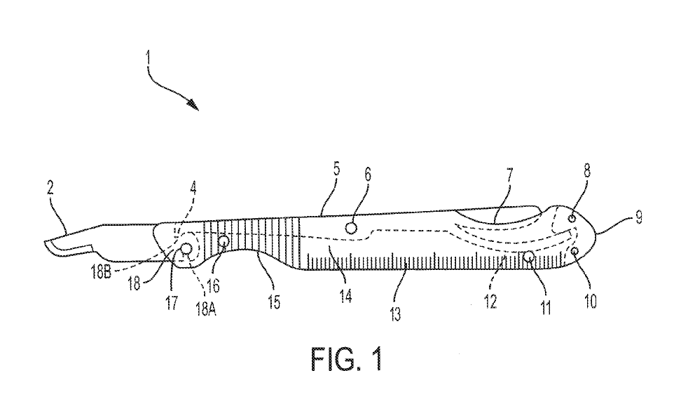

Referring to FIG. 1, a cutting device, such as a surgical scalpel 1, has a newly designed detachable blade 2 with a first end with a cutting surface and a second end configured to he quickly and safely attached onto reusable handles 14. A cavity is formed between the handles 14 into which a lever 5 and an elastic member 12 such as a spring (the elastic member 12 can be part of lever 5 or separate from lever 5) for activation of a locking mechanism 4, such as a cleat, are placed. A butt plate 9 is also placed in the cavity. The blade 2 has a unique attachment end 18 that allows the blade 2 to be attached to the handles 14 and detached from the handles 14 with a rotatable movement B, for example, 90 degrees (see FIG. 11). The two separate handles 14 are attached with a retaining pins/screws 11, 16 and 17 enabling easy breakdown for cleaning.

The handles 14 have a thumb groove 7 at the distal end on the top side. This allows the user to have contact with, and press down, the lever 5. The lever 5 has a lever pin 6 centrally located and the lever 5 can be pressed against the elastic member 12. The elastic member 12, which may be part of the lever 5 at the distal end, is located interior to the thumb groove 7 and can be held in place in a retention groove in butt plate 9. There is a retainer pin/screw 16 near the blade end of the handles 14 that holds the lever arm and handles 14 in place when the blade 2 is removed from the knife. The retainer pin/screw 16 also acts as a lever stop when the blade 2 is engaged into the end of the handles 14.

The blade 2 has an attachment end 18 so that it may be affixed to a retainer pin/screw 17 and can be locked into position by the lever locking cleat 4. In one embodiment, the attachment end 18 has an opening 18A sized such that the retainer pin/screw 17 can be inserted. In addition, the attachment end 18 may have a notch 18B, into which the lever locking cleat 4 can be inserted, to lock the blade in place. Part of the attachment end 18 may also be rounded to facilitate rotation of the blade 2 in the handles 14. The lever locking mechanism 4 may be L-shaped, or any other shape that can lock the blade in place.

The retainer pin/screw 16 also stops the lever 5 from falling down between the handles 14 when the blade 2 is removed. The lever pin 6 can be a self-locking implanted cotter (SLIC) pin, or other suitable pin/screw. This allows the handles 14 of the scalpel 1 to be taken apart to be cleaned and sterilized.

Note that in one embodiment, a measuring scale 13 can be marked on one or both of the handles 14 to assist the surgeon in making measurement. In addition, a finger grip 15 may be provided to make it easier for the surgeon to hold the scalpel 1. The grip can be notches scored in the handle or another suitable structure for making it easier to hold the scalpel and prevent slipping.

The blade packaging 20 is designed to expose the attachment end of he blade, or tang, while keeping the cutting edge covered. The blade 2 can be returned to the packaging 20 after use. This allows for easy assembly/disassembly of the blade 2 and handles 14. The blade packaging 20 can be constructed of recyclable material for quick, efficient and safe disposal.

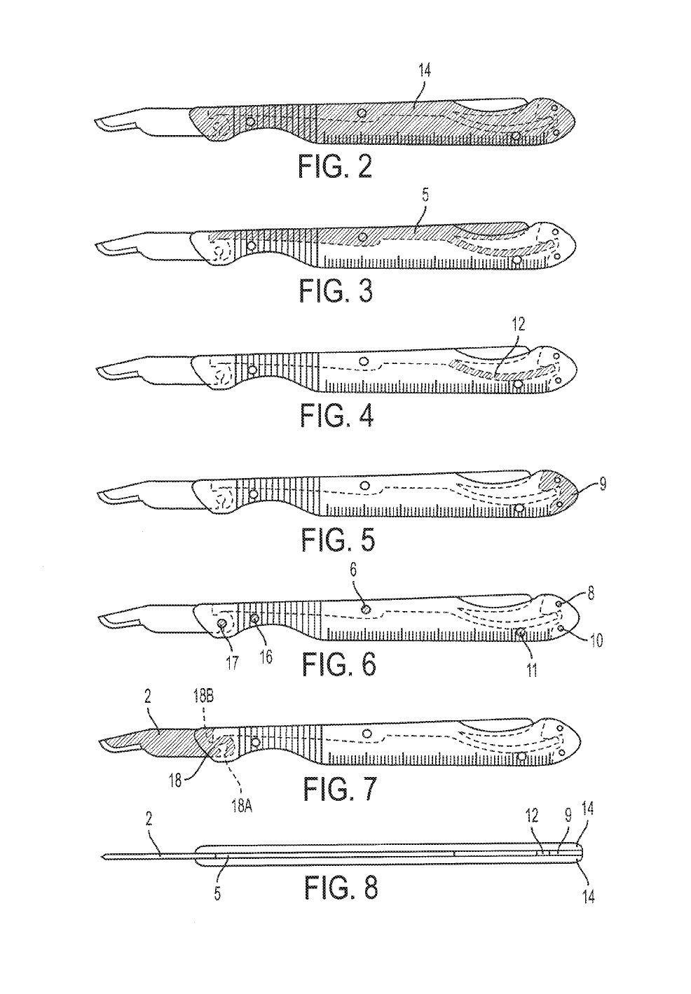

FIG. 2 is a side view of an exemplary embodiment of the scalpel 1 of the present invention with one of the handles 14 darkened to show its relative position to the other working parts.

FIG. 3 is a side view of an exemplary embodiment of the scalpel 1 of the present invention with the lever arm 5 darkened to show its relative position to the other working parts. The lever 5 could be made of stainless steel or titanium, or other suitable material.

FIG. 4 is a side view of an exemplary embodiment of the scalpel 1 of the present invention with the elastic member 12 darkened to show its relative position to the other working parts. As noted above, the elastic member 12 can be part of lever 5, or a separate component. The elastic member 12 could be made of stainless steel or titanium, or other suitable material.

FIG. 5 is a side view of an exemplary embodiment of the scalpel 1 of the present invention with the butt plate 9 darkened to show its relative position to the other working parts.

FIG. 6 is a side view of an exemplary embodiment of the scalpel 1 of the present invention with the retainer pin/screws 17, 16 and 11, and butt plate fixation screws 8, 10 darkened to show relative position to the other working parts.

FIG. 7 is a side view of an exemplary embodiment of the scalpel 1 of the present invention with the blade 2 darkened to show its relative position to the other working parts. The blade 2 may have many different types of cutting surfaces.

FIG. 8 is a top view of an exemplary embodiment of the scalpel 1 of the present invention. The handles 14 could be made of high heat zytel, G10 or stainless steel, or other suitable materials.

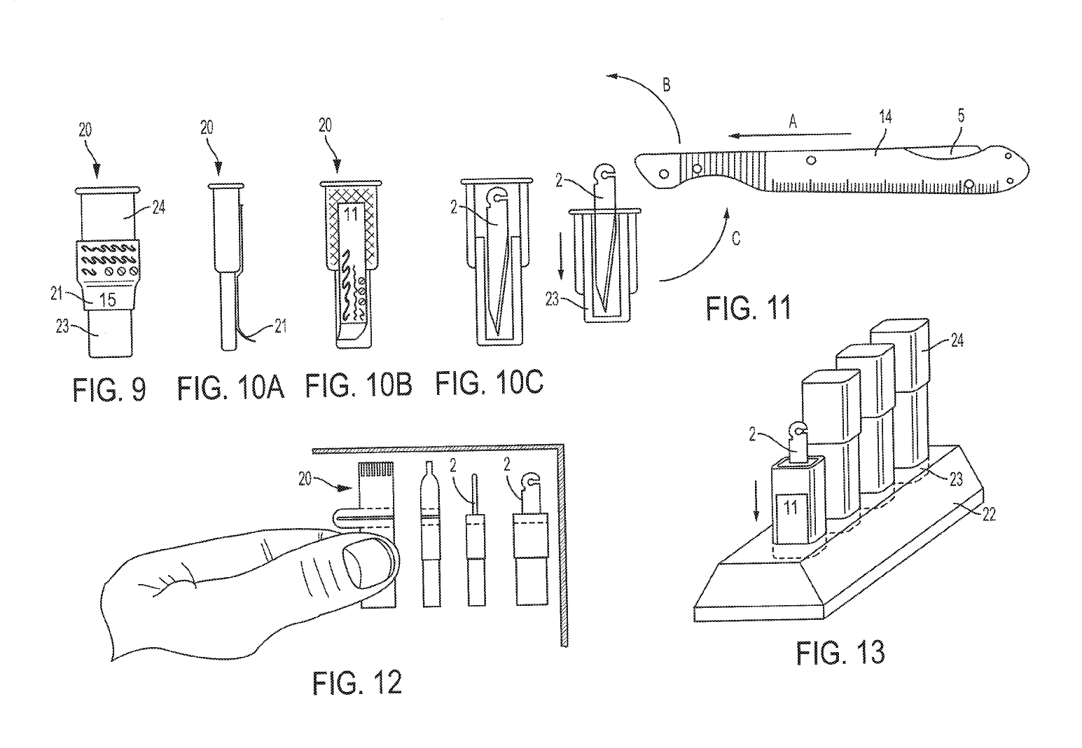

FIG. 9 is a side view of an exemplary embodiment of packaging 20 for a blade 2 of the present invention. A pull tab 21 can be wrapped around the packaging 20 horizontally or vertically. The pull tab 21 can also identify type of blade in the package.

FIGS. 10A, 10B and 10C are side views of the exemplary packaging 20. FIG. 10C shows the blade 2 in the packaging 20.

FIG. 11 shows an example of how the blade 2 can be attached to the handle 14, without the cutting edge of the blade 2 being exposed to the user. After the top 24 of the packaging 20 is removed, the distal end of the lever 5 is pressed so that the distal end moves down to the level of the thumb groove 7. This causes the lever 5 to pivot about the lever pin 6 causing the lever locking mechanism 4 to move up and away from the handles 14. The handles 14 are then moved in a perpendicular direction A toward the blade 2 such that the retainer pin/screw 17 is inserted into the opening 18A of attachment end 18. Note that depending on the location of the opening 18A, the handles do not necessarily need to be moved in a perpendicular direction in order to be inserted in opening 18A. Then the lever 5 is released causing the lever locking mechanism 4 to press against the attachment end 18. The handles 14 are then rotated 90 degrees in direction B. This causes the lever locking mechanism 4 to be inserted into notch 18B and lock the blade 2 in place. Note that depending on the location of the opening 18A, after the handle is inserted, other degrees of rotation may be used to lock the blade 2 in place.

In another embodiment, the blade 2 can be rotated 90 degrees in direction C to fold the blade into the handles 14, similar to a folding pocket or hunting knife.

The blade 2 can be removed by inserting the blade 2 into an empty packaging 20 and pressing the distal end of the lever 5 to release the locking mechanism 4 from notch 18B, rotating the handles 90 degrees and then pulling the handles 14 away from the blade 2 so that the retainer pin/screw 17 moves out of the opening 18A. This design allows the blade to be attached and removed without the user touching the blade 2.

FIG. 12 shows side views of the closed packaging 20 and views of the open packaging 20 that show the exposed blade tang.

FIG. 13 shows perspective views of closed and open packaging 20 with the bottom 23 of the packaging 20 mounted in a block 22. The bottom 23 of the packaging 20 can be smaller than the top so that the packages can be mounted in an upright position. The newly designed blade holder 22 allows for safe assembly/disassembly and disposal of the blade 2.

Although exemplary embodiments of the disclosure have been shown and described, it would be appreciated by those skilled in the art that changes may be made in these exemplary embodiments without departing from the principles and spirit of the exemplary embodiments, the scope of which is defined in the claims and their equivalents. For example, while one use of the invention is in the medical field as a surgical scalpel, the same principles could be used to make devices for other medical and non-medical fields.

What is claimed is:

1. A cutting device, comprising:
a blade with a first end with a cutting surface and a second end configured to be attached to a handle;
wherein said blade can be attached to said handle without a user contacting said blade; and
wherein said blade is configured to be locked in place in said handle by rotating said handle.

2. The cutting device of claim 1 wherein said second end of said blade comprises an opening in which said handle can be inserted.

3. The cutting device of claim 2 wherein said second end of said blade comprises a notch which can be used to lock said blade in said handle.

4. The cutting device of claim 3 further comprising a handle.

5. The cutting device of claim 4 wherein said handle comprises a lever configured to lock in place said blade in said handle.

6. The cutting device of claim 5 wherein said lever comprises an elastic member.

7. The cutting device of claim 6 wherein said lever comprises a lo mechanism configured to lock in place said blade in said handle.
